(19) United States
(12) Patent Application Publication
(10) Pub. No.: US 2002/0142304 A1
(43) Pub. Date: Oct. 3, 2002

(54) Uses and methods of making microarrays of polymeric biomaterials

(76) Inventors: Daniel G. Anderson, Framingham, MA (US); Robert S. Langer, Newton, MA (US); David A. Putnam, Cambridge, MA (US)

Correspondence Address: Sam Pasternack, Choate, Hall & Stewart, 53 State Street, Boston, MA, 02109, US

(21) Appl. No.: 09/803,319

(22) Filed: Mar. 9, 2001

(51) Int. Cl. C12Q 1/68; G01N 33/53; G01N 33/542; C12M 1/34

(52) U.S. Cl. 435/6; 435/7.1; 435/7.9; 435/287.2

(57) ABSTRACT

A microarray of polymeric biomaterials is provided. Specifically, a microarray of polymeric biomaterials that comprises a base with a cytophobic surface, and a plurality of discrete polymeric biomaterial elements bound to the cytophobic surface, is provided. Preferably said polymeric biomaterials comprise a synthetic polymer. Said polymeric biomaterials may also comprise other compounds covalently or non-covalently attached to said synthetic polymer. Methods of preparing the microarray of polymeric biomaterials of the present invention and uses of the microarray of polymeric biomaterials of the present invention are also provided.

### FIELD OF THE INVENTION

The present invention relates to high throughput screening methods, and more particularly, to high throughput screening methods that permit microarrayed polymeric biomaterials to be screened simultaneously for their ability to affect cellular behavior.

### BACKGROUND

The ability to control cellular behavior (e.g., adhesion, proliferation, differentiation, gene expression, etc.) would offer the potential for broad applications in basic and applied research. One way to affect cellular behavior is to modify the local environment in which a cell grows. Indeed, for cells that attach to surfaces, the chemical and physical properties of the surfaces to which they attach can greatly affect cellular behavior. In this context, a number of so called “biomaterials” and, in particular, polymeric biomaterials have recently been developed that, for example, promote or inhibit the adhesion and proliferation of a variety of cell types. For a review of current issues in the development of polymeric biomaterials and tissue engineering, see, for example, “Tissue Engineering” by Robert Langer in Molecular Therapy 1: 12, 2000; “The Importance of Drug Delivery Systems in Tissue Engineering” by Yasuhiko Tabata in Pharmaceutical Science and Technology Today 3:80, 2000; and “Biomaterials in Tissue Engineering” by Jeffrey Hubbell in Biotechnology 13:565, 1995; all of which are incorporated herein by reference.

Specific examples of some of the most recent developments in this area include, amongst others, an investigation of the attachment, proliferation, morphology, and differentiation of skeletal muscle cells and chondrocytes grown on different compositions of segmented block copolymers of poly(ethylene glycol) and poly(butylene terephthalate) (Papadaki et al., Journal of Biomedical Materials Research 54:47, 2001); an examination of the effect of polylysine on the proliferation of myelin-forming Schwann cells grown on glutaraldehyde cross-linked hyaluronic acid (Min et al., Tissue Engineering 6:585, 2000); and a comparison of the cellular growth and patterns of gene expression of smooth muscle cells grown on poly(glycolic acid) and type I collagen scaffolds (Kim et al., Experimental Cell Research 251:318, 1999).

As the above examples illustrate, investigations into the effects of polymeric biomaterials on cellular behavior are traditionally performed using specific combinations of polymeric biomaterials and cells. However, the number of polymeric biomaterials, cell types, and aspects of cellular behavior that could potentially be investigated is vast and continually expanding.

Accordingly, it is desirable to provide a method that would facilitate the high throughput screening of an extensive number of polymeric biomaterials for their ability to affect cellular behavior. In particular, it is desirable to provide a generalized method of forming microarrays of polymeric biomaterials, that could be used in combination with a variety of cell-based assays to screen for desirable interactions between a wide range of polymeric biomaterials and a wide range of cell types.

### SUMMARY OF THE INVENTION

In one aspect of the present invention, a microarray of polymeric biomaterials is provided. More specifically, a microarray that comprises a base with a cytophobic surface and a plurality of discrete polymeric biomaterial elements bound to the cytophobic surface is provided.

In another aspect of the present invention, a method of making a microarray of polymeric biomaterials is provided. This method comprises the steps of (1) providing a base with a cytophobic surface, (2) providing polymeric biomaterials as stock solutions in a suitable solvent, (3) depositing the polymeric biomaterials as discrete elements of a microarray on the cytophobic surface, and (4) removing the solvent by drying the microarray in a vacuum.

In preferred embodiments, the cytophobic surface is formed by coating a base with a hydrogel that has a low cell binding affinity. The base preferably comprises a material selected from the group consisting of glass, plastic, metal, and ceramic. The hydrogel is preferably selected from the group consisting of homopolymers of methacrylic acid esters, homopolymers of alkylene oxides, homopolymers of alkylene glycols, copolymers thereof, adducts thereof, and mixtures thereof.

In preferred embodiments, the polymeric biomaterial elements of the microarray comprise a synthetic polymer. The synthetic polymer may be selected from the group consisting of polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, poly(vinyl chloride), poly(vinyl fluoride), poly(vinyl imidazole), poly(vinyl alcohol), and chlorosulphonated polyolefins. In one embodiment, the polymeric biomaterials may comprise copolymers of these synthetic polymers. In another embodiment, the polymeric biomaterials may comprise adducts of these synthetic polymers. In yet another embodiment of the present invention, the polymeric biomaterials may comprise mixtures of these synthetic polymers.

In certain embodiments, the polymeric biomaterial elements of the microarray may also comprise a compound. The compound may be natural or synthetic. In certain embodiments, the compound may be covalently bound to the synthetic polymer component or components of the polymeric biomaterial. In other embodiments, the compound may be non-covalently bound to the synthetic polymer component or components of the polymeric biomaterial. Examples of natural compounds that may be used in the present invention include growth factors, proteins, polysaccharides, polynucleotides, lipids, copolymers of these, adducts of these, and mixtures of these. Examples of synthetic compounds that may be used in the present invention include literally any synthetic drug or combinatorial compound.

In a preferred embodiment, the polymeric biomaterial elements of the microarray are deposited on the cytophobic surface using a robotic liquid handling device. The robotic liquid handling device may, for example, use pin fluid deposition or ink-jet fluid deposition. Once they have been deposited, the polymeric biomaterials may become bound to the cytophobic surface via a variety of interactions such as, for example, chemical adsorption, hydrogen bonding, surface interpenetration, ionic bonding, covalent bonding, van der Waals forces, hydrophobic interactions, magnetic interactions, dipole-dipole interactions, or combinations of these.

A further aspect of the present invention includes a method of using the microarray of polymeric biomaterials to screen polymeric biomaterials for their ability to affect cellular behavior, the method comprising the steps of (1) seeding the microarray of polymeric biomaterials with cells, (2) allowing the cells to adhere to the polymeric biomaterials, and (3) assaying the cellular behavior of the cells attached to each of the polymeric biomaterial elements of the microarray.

The invention employs a wide range of cell types and is not limited to any specific cell type. The cells may, for example, be mammalian cells, bacterial cells, yeast cell, or plant cells. The invention also employs a wide range of cell-based assays and is not limited to any specific assay. The present invention may be used to investigate the effect of a variety of polymeric biomaterials on a variety of aspects of cellular behavior. Alternatively, the present invention may be used to investigate the effect of a variety of natural and synthetic compounds such as drugs, growth factors, combinatorial compounds, proteins, polysaccharides, polynucleotides, lipids, adducts thereof, and mixtures thereof on aspects of cellular behavior. Aspects of cellular behavior that may be investigated according to the present invention include, for example, cellular adhesion, cellular proliferation, cellular differentiation and gene expression.

### DESCRIPTION OF THE DRAWING

FIG. 1a depicts a top view of a microarray of polymeric biomaterials.

FIG. 1b depicts a side view of a microarray of polymeric biomaterials.

FIG. 2 is a photograph of a microarray of polymeric biomaterials.

FIG. 3 is a phase contrast photomicrograph of bovine chondrocyte cells growing on a single spot of a seeded microarray of polymeric biomaterials.

FIG. 4 is a fluorescence photomicrograph of immunostained bovine chondrocyte cells grown on a microarray of polymeric biomaterials.

FIG. 5 is a fluorescence photomicrograph of immunostained neural stem cells grown on a microarray of polymeric biomaterials.

### DESCRIPTION OF CERTAIN PREFERRED EMBODIMENTS

The present invention relates in general to the production of microarrays of polymeric biomaterials and to the uses of these microarrays of polymeric biomaterials. In one aspect, the present invention involves the steps of providing a substrate surface with a low cell binding affinity, providing the polymeric biomaterials, and arranging the polymeric biomaterials as elements of a microarray on the substrate surface. In another aspect, the present invention further involves the steps of seeding the microarray of polymeric biomaterials with cells and assaying the cellular behavior for each element of the microarray. These steps can be performed generally to screen for desirable interactions between a variety of polymeric biomaterials and a cell type of interest. The nature of the substrate surface, the nature of the polymeric biomaterials, the characteristics of the microarray, the nature of the cells and the details of the cell-based assay may be determined by the user. Certain examples of preferred substrate surfaces, polymeric biomaterials, microarrays, cell types, and cell-based assays are presented below. These examples are intended to clarify but not limit the present invention.

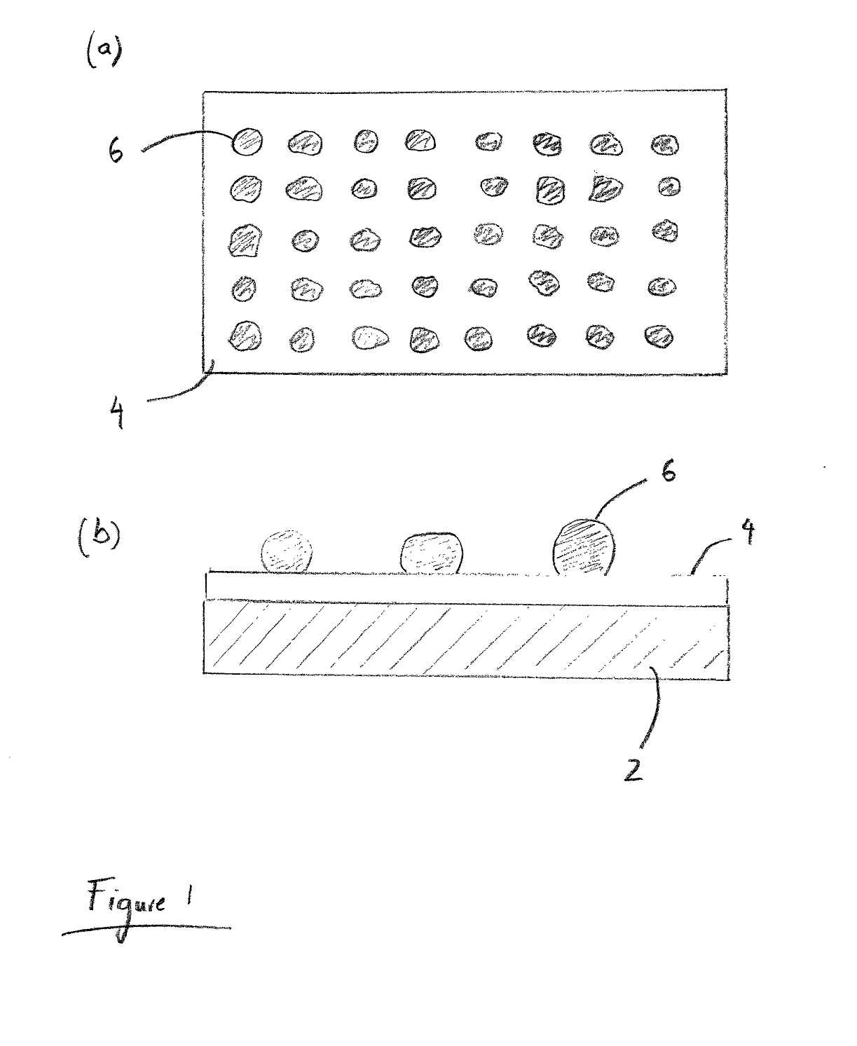

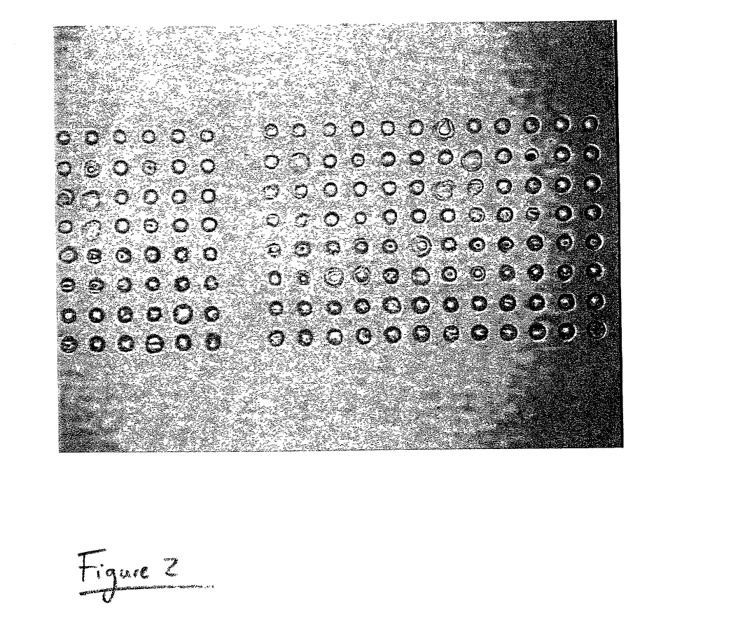

In one embodiment, the microarray of polymeric biomaterials of the present invention comprises a base 2 that is treated to produce a substrate surface with a low cell binding affinity 4 (a so-called “cytophobic” surface) across which are dispersed at regular intervals polymeric biomaterial elements 6 (FIGS. 1a and 1b). The cytophobic surface ensures that cell adhesion is limited to the polymeric biomaterial elements 6 of the microarray. The polymeric biomaterial elements 6 are preferably associated with the substrate surface 4 via non-covalent interactions such as chemical adsorption, hydrogen bonding, surface interpenetration, ionic bonding, van der Waals forces, hydrophobic interactions, magnetic interactions, dipole-dipole interactions, and combinations of these; however, the polymeric biomaterial elements 6 may also be associated with the substrate surface 4 via covalent interactions. The base 2 can be a glass, plastic, metal, or ceramic, but can also be made of any other suitable material. In a preferred embodiment, the base 2 is coated with a hydrogel that has a low cell binding affinity. A hydrogel is defined as a substance formed when an organic polymer (natural or synthetic) is cross-linked via covalent, ionic or hydrogen bonds to create a three-dimensional open-lattice structure that entraps water molecules to form a gel. The hydrogel may interact with the base 2 non-covalently (e.g., through hydrogen bonds, ionic bonds, van der Waals forces, magnetic interactions, etc., including combinations of these) or may be covalently attached to the base. In one embodiment, the base 2 may be modified to enhance its interaction with the coated hydrogel. An example of a modified base would be an epoxy modified glass, for example a light microscope slide or coverslip (e.g., XENOSLIDE™ E available from Xenopore Corp. of Hawthorne, N.J.). The surface of the base 2 is preferably rectangular in shape, with dimensions of about 25 mm by 75 mm, and the base is preferably 1 mm thick; however, the base 2 can be of any shape, and may be larger, smaller, thinner or thicker, as chosen by the practitioner.

A variety of hydrogels that have a low cell binding affinity are known in the art. In general, these polymers include unsaturated hydrocarbons and polar but uncharged groups, and are at least partially soluble in water or aqueous alcohol solutions. Examples of polymeric hydrogels that have a low cell binding affinity and may be used in the present invention include but are not limited to homopolymers and copolymers of methacrylic acid esters, alkylene oxides, and alkylene glycols.

As used herein, the term poly(methacrylic acid ester) refers to a polymer of the formula—[CH₂C(CH₃)(COOR)]ₓ—, wherein R refers to a C₁ to C₅ straight or branched chain alkyl or hydroxy substituted alkyl, including but not limited to methyl, ethyl, propyl, isopropyl, butyl, isotbutyl, pentyl, isopentyl, and their hydroxy substituted derivatives. X is an integer greater than 4, and typically between 8 and 400, and more preferably between 30 and 400. Specific examples of poly(methacrylic acid esters) that may be used in the present invention include but are not limited to poly(methyl methacrylate), poly(isobutyl methacrylate), poly(pentyl methacrylate), and poly(2-hydroxy-ethyl methacrylate). Preferred poly(methacrylic acid esters) include poly(2-hydroxy-ethyl methacrylate), often referred to as polyHEMA, and block copolymers comprising 2-hydroxy-ethyl methacrylate and one or more of methyl methacrylate, isobutyl methacrylate, and pentyl methacrylate. The properties and preparation of poly(methacrylic acid ester) hydrogels are discussed in detail in the literature. See, for example, Folkman et al., Nature 273:345, 1978; see also U.S. Pat. No. 5,266,325 to Kuzma, both of which are incorporated herein by reference.

As used herein, the term poly(alkylene oxide) (or poly(alkylene glycol) if the polymer was prepared from a glycol instead of an oxide) refers to a polymer of the formula—[(alkyl)O]_{y}—, wherein alkyl refers to a C₁ to C₄ straight or branched chain alkyl moiety, including but not limited to methyl, ethyl, propyl, isopropyl, butyl, and isobutyl. Y is an integer greater than 3, and typically between 8 and 500, and more preferably between 40 and 500. Specific examples of poly(alkylene oxides) and poly(alkylene glycols) that may be used in the present invention include but are not limited to poly(ethylene oxide), poly(propylene 1,2-glycol), and poly(propylene 1,3-glycol). Block copolymers of ethylene oxide and propylene oxide available commercially from BASF Corporation under the trademarked name PLURONIC™ may also be used in the present invention. Preferred members of the PLURONIC™ family of block copolymers include F68, F77, F87, F88, F98, F108, and F127. The preparation and properties of poly(alkylene oxide) hydrogels are discussed in detail in the literature; see, for example, Birch et al., Anal. Chem. 62:1123, 1990; Malmsten et al., Langmuir 7:2412, 1991; Lopez et al., J. Biomed. Mater. Res. 26:415, 1992; Sheu et al., J. Adhesion Sci. Tech. 7:1065, 1993; Merrill, J. Biomater. Sci. Polymer. Edn. 5:1, 1993; Johnston et al., in Plasma Treatments and Depositions of Polymers, Ed. by R. d'Agostino, Kluwer Academic Publishers, Dordrecht, The Netherlands, 1997; see also, U.S. Pat. No. 5,578,325 to Domb; all of which are incorporated herein by reference.

The base may be coated with the hydrogel by dip coating, spray coating, brush coating, roll coating, or spin casting. For example, the base may be coated with the hydrogel by dipping the base in an aqueous or aqueous-based solution of the hydrogel. An example of dip coating a hydrogel onto a base is described in greater detail in Example 1. In all of the above processing approaches, a suitable crosslinking agent may be incorporated to enhance the mechanical rigidity of the hydrogel. Divinyl benzene (DVB) and ethylene glycol dimethacrylate (EDMA) are non-limiting example of crosslinking agents that could be used to crosslink the polymer chains of a hydrogel. The hydrogel may also be coated on the base as a thin film of oligomers by radiofrequency (RF) plasma deposition. RF plasma deposition is a one step gas phase (i.e., dry) process and is reviewed in great detail in Ratner et al., J. Molec. Recogn. 9:617, 1996; Chinn et al., J. Tiss. Cult. Method. 16:155, 1994; Heshmati et al., Colloque de Physique 4:285, 1990; and Ratner et al., in Plasma Deposition, Treatment and Etching of Polymers, Ed. by R. d'Agostino, Academic Press, San Diego, Calif., 1990; all of which are incorporated herein by reference. As described in Lopez et al., J. Biomed. Mater. Res. 26:415, 1992, RF plasma deposition can, for example, be used to deposit oligomers such as triethylene glycol dimethyl ether or tetraethylene glycol dimethyl ether to form thin poly(ethylene oxide)-like thin films that have a low cell binding affinity.

Once the substrate surface of the microarray of the invention has been provided, it will be appreciated by one of ordinary skill in the art that a variety of polymeric compositions can be utilized to form the polymeric biomaterial elements of the microarray. In the present invention, the polymeric biomaterials are initially provided as stock solutions. Examples of solvents that may be used to prepare the stock solutions of the present invention include but are not limited to dimethylformamide, dimethylsufoxide, chloroform, dichlorobenzene, and other chlorinated solvents.

Preferably, the polymeric biomaterials of the present invention comprise at least one synthetic polymer. A number of biodegradable and non-biodegradable synthetic polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference). The term biodegradable, as used herein, refers to materials that are enzymatically or chemically (e.g., hydrolytically) degraded in vivo into simpler chemical species.

Biodegradable synthetic polymers that may be used in the present invention include but are not limited to polyamides, polyphosphazenes, polypropylfumarates, synthetic poly(amino acids), polyethers, polyacetals, polycyanoacrylates, biodegradable polyurethanes, polycarbonates, polyanhydrides, poly(ortho esters), polyhydroxyacids, and other biodegradable polyesters.

Non-biodegradable synthetic polymers that may be used in the present invention include but are not limited to polyacrylates, ethylene-vinyl acetate polymers and other cellulose acetates, polystyrenes, non-biodegradable polyurethanes, poly(vinyl chloride), poly(vinyl fluoride), poly(vinyl imidazole), poly(vinyl alcohol), non-biodegradable polyesters, and chlorosulphonated polyolefins.

In one embodiment, block copolymers or graft copolymers of the above biodegradable and non-biodegradable synthetic polymers may be used in the present invention.

In a preferred embodiment of the present invention, the synthetic polymers of the present invention are soluble in dimethylformamide, dimethylsulfoxide, chloroform, dichlorobenzene, methylene chloride, or some other chlorinated solvent at a concentration of at least 1 mg/ml, more preferably at least 5 mg/ml, most preferably at least 10 mg/ml.

Those skilled in the art will know how to synthesize the above polymers (see, for example, Concise Encyclopedia of Polymer Science and Polymeric Amines and Ammonium Salts, Ed. by Goethals, Pergamon Press, Elmsford, N.Y., 1980; Principles of Polymerization by Odian, John Wiley & Sons Inc., New York, N.Y., 1991; and Contemporary Polymer Chemistry by Allcock et al., Prentice-Hall Inc., Englewoods Cliffs, N.J., 1981; all of which are incorporated herein by reference) or may acquire them commercially (e.g., from Sigma, Union Carbide Corporation, ICI Group, DuPont Corporation, 3M Company, BASF Corporation, Dow Chemical Company, etc.). However, below we describe the preparation and properties of certain synthetic polymers that may be used in the present invention, namely biodegradable polyhydroxyacids and polyanhydrides. These examples are descriptions of certain embodiments of the present invention and are not intended to limit the scope of the invention as a whole.

Examples of polyhydroxyacids that may be used in the present invention include but are not limited to poly(lactic acid) (PLA), poly(glycolic acid) (PGA), polycaprolactone (PCL), and block copolymers of hydroxyacids such as poly(lactide-co-glycolide) (PLG), poly(lactide-co-caprolactone) (PLC), and poly(glycolide-co-caprolactone) (PGC) all of which are available commercially (e.g., from Sigma). The biodegradation of the above polyhydroxyacids is related in part to the molecular weights of the PLA, PGA and PCL polymers, or the PLG, PLC and PGC block copolymers. The higher molecular weights (e.g., weight average MW≧90,000 Da) result in polymeric biomaterials that retain their structural integrity for longer periods of time.

PLA polymers are usually prepared from the cyclic esters of lactic acids. Both L(+) and D(−) forms of lactic acid can be used to prepare the PLA polymers, as well as the optically inactive DL-lactic acid mixture. Methods of preparing polylactides are well documented in the patent literature. The following U.S. Patents, the teachings of which are hereby incorporated by reference, describe in detail polylactides, their properties and their preparation: U.S. Pat. Nos. 3,531,561; 2,683,136 to Trehu; U.S. Pat. No. 2,951,828 to Zeile; U.S. Pat. No. 2,758,987 to Salzberg; U.S. Pat. No. 2,703,316 to Schneider; U.S. Pat. No. 2,676,945 to Higgins; and U.S. Pat. No. 1,995,970 to Dorough. PGA is the homopolymer of glycolic acid. In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in “Cyanamid research develops world's first synthetic absorbable suture”, Chemistry and Industry 905, 1970. PCL polymers are usually prepared from the cyclic esters of lactones in the presence of rare earth metal catalysts such as yttrium. Methods of preparing polycaprolactones are well documented in the patent literature. U.S. Pat. Nos. 5,028,667 and 5,095,098 to McLain, the teachings of which are hereby incorporated by reference, describe in detail polycaprolactones, their properties and their preparation. A variety of methods are also known in the art that can be used to produce copolymers of hydroxyacids and other monomers. For example, U.S. Pat. No. 5,578,325 to Domb (incorporated herein by reference) discloses a method of making non-linear hydrophilic-hydrophobic multiblock copolymers comprising a hydrophilic alkylene glycol polymer and a hydrophobic polymer such as one of the polyhydroxyacids described above.

Examples of polyanhydride polymers that may be used in the present invention include but are not limited to block copolymers composed of sebacic acid, adipic acid, isophthalic acid, bis(p-carboxyphenoxy) methane, bis(p-carboxyphenoxy) propane, bis(p-carboxyphenoxy) hexane, 1,4-phenylene dipropionic acid and dodecanedioic acid. Polyanhydrides possess a water labile linkage and a hydrophobic backbone. The anhydride bond is hydrolytically more active than the ester bond of polyhydroxyacids such as PLA, PGA, and PCL. Polyanhydrides are traditionally prepared by the reaction of a dicarboxylic acid with an excess of acetic anhydride or acetyl chloride. Aromatic polyanhydrides are more stable hydrolytically than aliphatic polyanhydrides and are synthesized by heating mixed aliphatic-aromatic anhydrides. Methods of preparing polyanhydrides are well documented in the patent literature. U.S. Pat. No. 5,122,367 to Ron and U.S. Pat. Nos. 5,019,379; 4,916,204; 4,857,311 and 4,757,128 to Domb, the teachings of which are hereby incorporated by reference, describe in detail polyanhydrides, their properties and their preparation. A variety of methods are also known in the art that can be used to produce copolymers of anhydrides and other monomers. For example, U.S. Pat. No. 5,010,167 to Ron (incorporated herein by reference) discloses a method of making poly(amide-co-anhydrides) and poly(imide-co-anhydrides) from amido- or imido-dicarboxylic acid monomers with other dicarboxylic acids such as sebacic acid. In vivo, the anhydride linkages are hydrolytically degraded and the internal imide and amide bonds of the dicarboxylic acid are enzymatically degraded.

In one embodiment of the invention, the polymeric biomaterials may consist of a single type of synthetic polymer. However, the polymeric biomaterials are not limited to individual synthetic polymers. For example, in certain embodiments, the polymeric biomaterials may comprise mixtures of at least two different types of synthetic polymer. Polymer mixtures (often referred to as blends or composites), have advantageous physical and mechanical properties not exhibited by the individual polymer components. The component synthetic polymers are held together by non-covalent intermolecular interactions such as hydrogen bonding, ionic bonding, magnetic interactions, interpenetration, dipole-dipole interactions, van der Waals forces, or combinations of these. Polymer mixtures can exist as miscible one-phase systems, as semimiscible systems with miscible domains that co-exist with phases rich in one of the constituent synthetic polymers, or as immiscible multi-phase polymer systems. In a miscible mixture, the interactions between the various components are presumably stronger than the interactions between the individual molecules of a single species. Miscible and semimiscible mixtures are preferred.

As will be appreciated by one of ordinary skill in the art, the polymeric composition of the polymeric biomaterials may be further modified in a variety of ways. For example, one could envisage preparing polymeric biomaterials comprising different molecular weight distributions of the component synthetic polymer or polymers. Alternatively, when two or more polymers are involved as a blend or as adducts, one could envisage preparing polymeric biomaterials comprising different ratios of the component synthetic polymers. Alternatively, or additionally, when copolymers are involved, one could envisage varying the ratio of the component monomers.

The preparation and properties of a variety of synthetic polymer mixtures have been described in the art. For example, in an effort to modify the morphology and biodegradation profile of poly(L-lactic acid), poly(L-lactic acid) has been blended with poly(D-lactic acid) (Cha et al., Biomaterials 11: 108, 1990); with poly(ethylene-vinyl acetate) (Dollinger et al., ACS Polymer Preprint 32:429, 1990); and with members of the PLURONIC™ family of polyether block copolymers (U.S. Pat. No. 5,330,768 to Park); all of which are incorporated herein by reference.

A variety of methods of preparing mixtures of synthetic polymers are known in the art. Mixtures can, for example, be prepared by mixing two or more synthetic polymers in an appropriate solvent or co-solvent. Examples of preferred solvents include but are not limited to dimethylformamide, dimethylsulfoxide, chloroform, dichlorobenzene, methylene chloride, and other chlorinated solvents in which the two or more synthetic polymers are soluble. Alternatively, as is well known in the art, mixtures of synthetic polymers can be prepared by melt mixing.

One aspect of the invention involves the recognition that the synthetic polymers of the polymeric biomaterials may be functionalized by incorporation of additional components. In one embodiment, natural compounds (as defined below) may be incorporated with the synthetic polymer component of the polymeric biomaterials. For example, as is well known in the art, the attachment, growth and differentiation of cells on synthetic polymers may be enhanced by incorporating certain natural compounds with the synthetic polymers. These include but are not limited to polypeptides and polypeptide derivatives such as glycoproteins, lipoproteins, hormones, antibodies, basement membrane components (e.g., laminin, fibronectin), collagen types I, II, III, IV, and V, albumin, gelatin, fibrin, and polylysine; polysaccharides and polysaccharide derivatives such as agar, agarose, gum arabic, and alginate; glycosaminoglycans such as heparin, heparin sulfate, chondroitin, chondroitin sulfate, dermatin, and dermatin sulfate; and polynucleotides such as genes, antisense molecules which bind to complementary DNA to inhibit transcription, ribozymes and ribozyme guide sequences. Natural compounds of the invention may also include immunomodulators, inhibitors of inflammation, regression factors, inducers of differentiation or de-differentiation, attachment factors, growth factors, and lipids. Examples of growth factors that may be used in the present invention include but are not limited to heparin binding growth factor (HBGF), alpha or beta transforming growth factor (α- or β-TGF), alpha fibroblastic growth factor (α-FGF), epidermal growth factor (EGF), vascular endothelium growth factor (VEGF), nerve growth factor (NGF) and muscle morphogenic factor (MMP). Examples of lipids that may be used in the present invention include but are not limited to L-alpha-phosphatidyl-L-serine, L-alpha-phosphatidyl-DL-glycerol, L-alpha-phosphatidic acid, L-alpha-phosphatidylcholine, L-alpha-lysophosphatidylcholine, sphingomyelin, and cardiolipin. Such compounds are well known in the art and are commercially available or described in the controlled drug delivery or tissue engineering literature.

In another embodiment, synthetic compounds (as defined below) may be incorporated with the synthetic polymer component of the polymeric biomaterials. Examples of compounds that can be present as components of the polymeric biomaterials of the microarray of the invention include but are not limited to drugs and combinatorial compounds. For example, one particularly attractive application of the present invention would involve using the microarray of polymeric biomaterials of the present invention to screen the compounds of any combinatorial library for novel effects on cellular behavior. In one embodiment, the compounds are drugs that have already been deemed safe and effective for use by the appropriate governmental agency or body. For example, drugs for human use listed by the Food and Drug Administration (FDA) under 21 C.F.R. §§330.5, 331-361, 440-460, and drugs for veterinary use listed by the FDA under 21 C.F.R. §§500-582, all of which are incorporated herein by reference, are all considered acceptable for use in the present inventive microarray of polymeric biomaterials. A more complete non-limiting listing of classes of synthetic compounds suitable for use in the present invention may be found in the Pharmazeutische Wirkstoffe Ed. by Von Kleemann et al., Stuttgart/New York, 1987, incorporated herein by reference.

The compound or compounds of the biomaterial may be present as adducts or as a mixtures with the synthetic polymer component or components of the biomaterial. A variety of methods are known in the art that enable the covalent attachment of compounds and synthetic polymers. For example, U.S. Pat. No. 5,654,381 to Hrkach (incorporated herein by reference) discloses a method of forming graft copolymers of polyhydroxyacids such as PLA and PGA and amino acids. According to the method disclosed therein, peptides possessing an RGD (arginine-glycine-aspartic acid) amino acid sequence may be attached to polyhydroxyacids. The RGD sequence, present in proteins such as fibronectin, has been shown to be active in promoting cell adhesion and proliferation (see Massia et al., J. Cell. Biol. 114:1089, 1991). Alternatively, as is well known in the art, the attached amino acid sequence may be used as a linker that can be used to attach a variety of the above described compounds.

The invention may employ a ligand/receptor type interaction to indirectly link a compound and a synthetic polymer of the invention. Any ligand/receptor pair with a sufficient stability and specificity to operate in the context of the inventive system may be employed. To give but one example, the compound may be linked or associated with biotin and the synthetic polymer with avidin (or streptavidin). The strong binding of biotin to avidin (or streptavidin) would then allow for association of the compound with the synthetic polymer. Other possible ligand/receptor pairs include antibody/antigen, FK506/FK506-binding protein (FKBP), rapamycin/FKBP, cyclophilin/cyclosporin, and glutathione/glutathione transferase pairs. Other ligand/receptor pairs are well known to those skilled in the art.

A variety of non-covalently bound combinations of synthetic polymers and compounds are also known in the art of tissue engineering and drug delivery. For example, U.S. Pat. No. 5,629,009 to Laurencin and U.S. Pat. No. 5,286,763 to Gerhart (both incorporated herein by reference) disclose biodegradable polymers such as polyanhydrides that incorporate compounds such as growth factors. In addition, the preparation of polymeric biomaterials comprising chemotherapeutic drugs is described in Walter et al., Neurosurgery 37:1129, 1195; comprising immunosuppressants is described in Katayama et al., Int. J. Pharm. 115:87, 1995; comprising anti-inflammatory agents is described in Conforti et al., J. Pharm. Pharmacol. 48:468, 1996; comprising antibiotics is described in Schierholz et al., Drug. Res. 47:70, 1997; comprising opiod antagonists is described in Falk et al., J. Controlled Release 44:77, 1997; and comprising steroids is described in Ye et al., J. Controlled Release 41:259, 1996; all of which are incorporated herein by reference. The synthetic polymers can be mixed with or used to encapsulate the compounds using methods known to those skilled in the art, including mixing synthetic polymer particles and compression, solvent casting, and microencapsulation within synthetic polymers.

Once the appropriate polymeric biomaterials and the substrate surface have been selected for use in the present invention, it will be appreciated that the polymeric biomaterials can be microarrayed in a variety of ways on the substrate surface using a range of techniques known in the art. In a preferred embodiment of the invention, the elements of the microarray of polymeric biomaterials are deposited on the cytophobic surface using an automated liquid handling device. A number of devices are commercially available, including but not limited to devices such as the SYNQUAD 5500™ liquid handling robot (available from Cartesian Technologies, Inc. of Irvine, Calif.). As mentioned above, the polymeric biomaterials of the invention are initially provided as stock solutions. Stock solutions of the polymeric biomaterials are prepared having a total biomaterial concentration (i.e., including the synthetic polymer or polymers and bound compound or compounds) that ranges from about 10 to about 200 mg/ml, preferably from about 20 to about 100 mg/ml, most preferably from about 30 to about 70 mg/ml. According to the present invention, the compound or compounds may be incorporated to between 0 and 40% by weight of the biomaterial. Once the stock solutions of the polymeric biomaterials have been prepared, a predetermined volume of each biomaterial stock solution is placed in the separate reservoirs of the robotic liquid handling device.

In one embodiment of the present invention, the elements of the microarray are formed by depositing small drops of each polymeric biomaterial stock solution at discrete locations on the substrate surface. In a preferred embodiment of the invention, the elements of the microarray are deposited on the substrate surface as drops that range in volume from 0.1 to 100 nl. Preferably the drops are 1 nl in volume; however, smaller and larger volumes may be deposited on the substrate surface. The deposited drops form elements on the substrate surface that have horizontal and vertical dimensions of between about 10 and 1000 μm, preferably between about 50 and 500 μm. The “horizontal dimension”, as that term is used herein, means the dimension of the element when viewed from a direction that is parallel to the substrate surface (i.e., from the side). The “vertical dimension”, as that term is used herein, means the dimension of the element when viewed from a direction that is perpendicular to the substrate surface (i.e., from above). Preferably, the dimensions of the elements of the microarray are substantially the same; however, in certain embodiments of the present invention, the dimensions of the elements of the microarray may differ from one element to the next. The vertical dimensions of elements of the microarray of the present invention are such that each element may comprise hundreds or even thousands of layers of polymer molecules. When viewed from above or from the side, the elements may be circular, oblong, elliptical, square or rectangular. Preferably, the overall shape of the elements is sphere-like or disk-like. In one embodiment, the drops are deposited at intervals that range from about 300 to about 1200 μm. In a preferred embodiment, the drops are deposited at about 500 μm intervals; however, the drops may be deposited at smaller or larger intervals. The elements of the microarray may be present at a density on the substrate surface that ranges from about 1 to about 1000 polymeric biomaterial elements per cm². Preferably, the elements of the microarray are present at a density on the substrate surface that ranges from about 10 to about 100 polymeric biomaterial elements per cm².

The drops may be deposited on the substrate surface using a microarray of pins (e.g., ChipMaker2™ pins, available from TeleChem International, Inc. of Sunnyvale, Calif.). A range of pins exist that take a sample volume up by capillary action and deposit a spot volume of 1 to 10 nl. In another embodiment, the drops may be deposited on the substrate surface using syringe pumps controlled by micro-solenoid ink-jet valves that deliver volumes greater than about 10 nl (e.g., using printheads based on the SYNQUAD™ technology, available from Cartesian Technologies, Inc. of Irvine, Calif.). Alternatively, the drops may be deposited on the substrate surface using piezoelectric ink-jet fluid technology that deposits smaller drops with volumes between about 0.1 and 1 nl (e.g., using the MICROJET™ printhead available from MicroFab Technologies, Inc. of Plano, Tex.).

In one embodiment, the drops are arranged as a rectangular microarray. The size of the array may be determined by the user and will depend on the size of the elements of the array, the spacing between the elements and the size of the substrate surface. The rectangular microarray may, for example, be an 18×40, an 18×54 or a 22×64 microarray; however, smaller, larger and alternatively shaped microarrays (e.g., square, triangular, circular, elliptical, etc.) may be used.

In one embodiment of the invention, each element of the microarray is formed by depositing a single drop taken from one of the polymeric biomaterial stock solutions. In another embodiment, some or all of the elements are formed by depositing at least two drops taken from one of the polymeric biomaterial stock solutions. In yet another embodiment, some or all of the elements are formed by depositing at least two drops taken from at least two different polymeric biomaterial stock solutions. It may be advantageous to layer the same or different polymeric biomaterials on a single element of the microarray. For example, one could envisage burying a polymer layer of interest within several biodegradable layers so that access to the layer of interest, or alternatively release of a compound from the layer of interest can be controlled. The use of biodegradable polymers for this purpose is well known in the art of tissue engineering and drug delivery.

One aspect of the present invention involves the recognition that an endless number of combinations of synthetic polymers and natural and/or synthetic compounds can be obtained according to the present invention by varying the compositions of the stock solutions that are initially added to the robotic liquid handling device and/or by layering drops taken from these stock solutions in a series of sequential deposition steps.

In one embodiment of the invention, once the complete microarray of elements has been deposited, the polymeric biomaterial microarray is placed in an evacuated desiccator at about 25° C. for 12 to 48 hrs to remove any residual solvent. In another embodiment of the invention, in particular when some of the elements of the array are formed by the deposition of at least two drops taken from the same or different polymeric biomaterial stock solutions, the residual solvent may be removed, as described above, in between individual deposition steps. Example 1 provides a description of the preparation of several microarrays of polymeric biomaterials.

In one embodiment of the present invention, the microarray of polymeric biomaterials provided above may be seeded with cells. The invention employs a wide range of cell types and is not limited to any specific cell type. Examples of cell types that may be used include but are not limited to bone or cartilage forming cells such as chondrocytes and fibroblasts, other connective tissue cells such as epithelial and endothelial cells, cancer cells, hepatocytes, islet cells, smooth muscle cells, skeletal muscle cells, heart muscle cells, kidney cells, intestinal cells, other organ cells, lymphocytes, blood vessel cells, and stem cells such as human embryonic stem cells or mesenchymal stem cells. For therapeutic applications, it is preferable to practice the invention with mammalian cells, and more preferably human cells. However, non-mammalian cells such as bacterial cells (e.g., E. Coli), yeast cells (e.g., Saccharamomyces Cerevisiae) and plant cells may also be used with the present invention.

The cells are first cultured in a suitable growth medium as would be obvious to one of ordinary skill in the art. See, for example, Current Protocols in Cell Biology, Ed. by Bonifacino et al., John Wiley & Sons Inc., New York, N.Y., 2000 (incorporated herein by reference). A microarray of polymeric biomaterials prepared as above is then placed in a suitable container (e.g., a 25 mm by 150 mm round suspension culture dish) and incubated with a solution of the cultured cells. Preferably the cells are present at a concentration that ranges from about 10,000 to 500,000 cells/cm³, although both higher and lower cell concentrations may be used. The incubation time and conditions (e.g., temperature, CO₂ and O₂ levels, growth medium, etc.) will depend on the nature of the cells that are under evaluation. For most cell types, the choice of conditions will be obvious to one skilled in the art. The incubation time should be sufficiently long to allow the cells to adhere to the elements of the polymeric biomaterial microarray. In one embodiment of the invention, the environmental conditions will need to be optimized in a series of screening experiments.

In a preferred embodiment of the invention, the cellular behavior of the seeded cells is assayed for each element of the microarray. The invention employs a wide range of cell-based assays that enable the investigation of a variety of aspects of cellular behavior. For the purposes of clarification only, and not for limitation, we discuss certain of these cell-based assays in more detail below.

Cell-based assays screen for interactions at the cellular level using cellular targets and are to be contrasted with molecular-based assays that screen for interactions at a molecular level using molecular targets. Although the sheer number of cellular components and the inherent complexity of cellular behavior can make the interpretation of cell-based assays somewhat complex, their scope, practical relevance and versatility is significantly greater than that of some of the simpler but more specific molecular assays. Indeed, by employing a cellular environment to screen for a given outcome (e.g., expression of a gene of interest) the experimenter does not require prior knowledge of the specifics of the interactions involved (e.g., the nature of the surface receptor or cytoplasmic cascade that triggers expression of the gene of interest). As a consequence, when used with an appropriate assay, the “black box” that is the cellular machinery can, amongst other things, dramatically simplify and shorten the screening process.

The cellular behaviors that can potentially be investigated according to the invention include but are not limited to cellular adhesion, proliferation, differentiation and gene expression. One may be interested in screening for polymeric biomaterials that promote or inhibit the adhesion of a given cell type. Alternatively or additionally, one may be interested in screening for polymeric biomaterials that enhance the proliferation of a given cell type. For example, polymeric biomaterials that enhance the adhesion and proliferation of chondrocytes could be used as scaffolds in the preparation of engineered cartilage. One may further be interested in screening for polymeric biomaterials that cause attached cells to differentiate or de-differentiate in a desirable way. More specifically, one may be interested in screening for polymeric biomaterials that promote or inhibit the expression of a given gene within a cell. For example, polymeric biomaterials that cause neural stem cells to differentiate into glial cells or neurons may be useful as scaffolds in the regeneration of neural tissue.

It will be appreciated that any of the cell-based assays known in the art may be used according to the present invention to screen for desirable interactions between the polymeric biomaterials of the microarray and a given cell type. When they are assayed, the cells may be fixed or living. Preferred assays employ living cells and involve fluorescent or chemiluminescent indicators, most preferably fluorescent indicators. A variety of fixed and living cell-based assays that involve fluorescent and/or chemiluminescent indicators are known in the art. For a review of cell-based assays, see Current Protocols in Cell Biology, Ed. by Bonifacino et al., John Wiley & Sons Inc., New York, N.Y., 2000; Current Protocols in Molecular Biology, Ed. by Ausubel et al., John Wiley & Sons Inc., New York, N.Y., 2000; Current Protocols in Immunology, Ed. by Coligan et al., John Wiley & Sons Inc., New York, N.Y., 2000; Sundberg, Curr. Opin. Biotechnol. 11:47, 2000; Stewart et al., Methods Cell Sci. 22:67, 2000; and Gonzalez et al., Curr. Opin. Biotechnol. 9:624, 1998; all of which are incorporated herein by reference.

Specific cell-based assays that can be used according to the present invention include but are not limited to assays that involve the use of phase contrast microscopy alone or in combination with cell staining; immunocytochemistry with fluorescent-labeled antibodies; fluorescence in situ hybridization (FISH) of nucleic acids; gene expression assays that involve fused promoter/reporter sequences that encode fluorescent or chemiluminescent reporter proteins; in situ PCR with fluorescently labeled oligonucleotide primers; fluorescence resonance energy transfer (FRET) based assays that probe the proximity of two or more molecular labels; and fused gene assays that enable the cellular localization of a protein of interest. The steps involved in performing such cell-based assays are well known in the art. For the purposes of clarification only, and not for limitation, certain properties and practical aspects of some of these cell-based assays are considered in greater detail in the following paragraphs.

Currently, fluorescence immunocytochemistry combined with fluorescence microscopy allows researchers to visualize biological moieties such as proteins or DNA within a cell (for a review on confocal microscopy, see Mongan et al., Methods Mol. Biol. 114:51, 1999; for a review on fluorescence correlated spectroscopy, see Rigler, J. Biotechnol. 41:177, 1995; and for a review on fluorescence microscopy, see Hasek et al., Methods Mol. Biol. 53:391, 1996; all of which are incorporated herein by reference). One method of fluorescence immunocytochemistry involves the first step of hybridizing primary antibodies to the desired cellular target. Then, secondary antibodies conjugated with fluorescent dyes and targeted to the primary antibodies are used to tag the complex. The complex is visualized by exciting the dyes with a wavelength of light matched to the dye's excitation spectrum. A variety of fluorescent dyes such as fluorescein and rhodamine are known in the art. Appropriate antibodies are well described in the art, and a variety of labeled and unlabeled primary and secondary antibodies are available commercially (e.g., from Sigma). Examples 2 and 3 provide descriptions of fluorescent immunocytochemistry assays for the detection of collagen II in chondrocytes and neurofilament in neural stem cells, respectively.

Colocalization of biological moieties in a cell may be performed using different sets of antibodies for each cellular target. For example, one cellular component can be targeted with a mouse monoclonal antibody and another component with a rabbit polyclonal antibody. These are designated as primary antibodies. Subsequently, secondary antibodies to the mouse antibody or the rabbit antibody, conjugated to different fluorescent dyes having different emission wavelengths, are used to visualize the cellular target. An ideal combination of dyes for labeling multiple components within a cell would have well-resolved emission spectra. In addition, it would be desirable for this combination of dyes to have strong absorption at a coincident excitation wavelength.

As will be appreciated by one of ordinary skill in the art, fluorescent immunocytochemistry can be used to assay for cellular adhesion, gene expression, and cell proliferation. In one embodiment, fluorescent molecules such as the Hoechst dyes (e.g., benzoxanthene yellow or DAPI (4,6-diamidino-2-phenylindole)) that target and stain DNA directly and non-specifically can be used to estimate the total cell population on each element of a seeded microarray of the invention. As is well known in the art, such estimates can be used to normalize the measured levels of a biological moiety of interest (e.g., an expressed protein) within the cells that are attached to the elements of a seeded microarray.

Fluorescence in situ hybridization (FISH) typically involves the fluorescent tagging of an oligonucleotide probe to detect a specific complementary DNA or RNA sequence. For a review of FISH see, Swiger et al., Environ. Mol. Mutagen. 27:245, 1996; Raap, Mut. Res. 400:287, 1998; and Nath et al., Biotechnic. Histol. 73:6, 1997; all of which are incorporated herein by reference. An alternative approach is to use an oligonucleotide probe conjugated with an antigen such as biotin or digoxygenin and a fluorescently tagged antibody directed toward that antigen to visualize the hybridization of the probe to its DNA target. A variety of FISH formats are known in the art. See, for example, Dewald et al., Bone Marrow Transplant. 12:149, 1993; Ward et al., Am. J. Hum. Genet. 52:854, 1993; Jalal et al., Mayo Clin. Proc. 73:132, 1998; Zahed et al., Prenat. Diagn. 12:483, 1992; Kitadai et al., Clin. Cancer Res. 1:1095, 1995; Neuhaus et al., Human Pathol. 30:81, 1999; Buno et al., Blood 92:2315, 1998; Patterson et al., Science 260:976, 1993; Patterson et al., Cytometry 31:265, 1993; Borzi et al., J. Immunol. Meth. 193:167, 1996; Wachtel et al., Prenat. Diagn. 18:455, 1998; Bianchi, J. Perinat. Med. 26:175, 1998; and Munne, Mol. Hum. Reprod. 4:863, 1998; all of which are incorporated herein by reference.

Fluorescence resonance energy transfer (FRET) provides a method for detecting the proximity of two or more biological compounds by detecting the long-range resonance energy transfer that can occur between two organic fluorescent dyes if the spacing between them is less than approximately 100 Å. Conversely, this effect can be used to determine that two or more biological compounds are not in proximity to each other. For reviews on FRET, see Clegg, Curr. Opin. Biotechnol. 6:103, 1995; Clegg, Methods Enzymol. 211:353, 1992; and Wu et al., Anal Biochem. 218:1, 1994; all of which are incorporated herein by reference.

Cell-based assays that use promoter/reporter genes are designed to assay for expression of a gene of interest. Typically, this is achieved by transforming a given cell type with a plasmid comprising the promoter region of the gene of interest fused to the reporter sequence of a fluorescent or chemiluminescent protein. If the cytoplasmic cascade that normally leads to expression of the gene of interest and involves binding of a promoter moiety to the promoter sequence of the gene of interest is triggered, the transformed cells will begin to produce the reporter protein. Reporter genes that are known in the art include the genes that code for the family of blue, cyan, green, yellow, and red fluorescent proteins; the gene that codes for luciferase, a protein that emits light in the presence of the substrate luciferin; and the genes that code for β-galactosidase and β-glucuronidase (proteins that hydrolyze colorless galactosides and glucuronides respectively to yield colored products). A variety of vectors that contain fused promoter/reporter genes are available commercially (e.g., from Clontech Laboratories, Inc. of Palo Alto, Calif.). Example 4 provides a description of a gene expression assay designed to detect the expression of a gene of interest.

In another aspect of the invention, methods and devices for analyzing the cell-based assays for each element of the polymeric biomaterial microarray are provided. The devices may be manually or automatically operated. For example, an automated device that detects multicolored luminescent indicators can be used to acquire an image of the microarray and resolve it spectrally. Without limiting the scope of the invention, the device can detect samples by imaging or scanning. Imaging is preferred since it is faster than scanning. Imaging involves capturing the complete fluorescent or chemiluminescent data in its entirety. Collecting fluorescent or chemiluminescent data by scanning involves moving the sample relative to the imaging device.

In one embodiment of the present invention, there are three parts to the device: 1) a light source, 2) a monochromator to spectrally resolve the image, or a set of narrow band filters, and 3) a detector array. The light source is only required for the detection of fluorescent indicators. In one embodiment, the light source may be derived from the output of a white light source such as a xenon lamp or a deuterium lamp that is passed through a monochromator to extract out the desired wavelengths. Alternatively, filters could be used to extract the desired wavelengths. In another embodiment, any number of continuous wave gas lasers can be used. These include, but are not limited to, any of the argon ion laser lines (e.g., 457, 488, 514 nm, etc.), a HeCd laser, or a HeNe laser. Furthermore, solid state diode lasers could be used.

To spectrally resolve two different fluorescent or chemiluminescent indicators, light from the microarray may be passed through an image-subtracting double monochromator. Alternatively, the fluorescent or chemiluminescent light from the microarray may be passed through two single monochromators with the second one reversed from the first. The double monochromator consists of two gratings or two prisms and a slit between the two gratings. The first grating spreads the colors spatially. The slit selects a small band of colors, and the second grating recreates the image.

In a preferred embodiment, the fluorescent or chemiluminescent images are recorded using a camera preferably fitted with a charge-coupled device (CCD). A CCD is a light sensitive silicon solid state device composed of many small pixels. The light falling on a pixel is converted into a charge pulse which is then measured by the CCD electronics and represented by a number. A digital image is the collection of such light intensity numbers for all of the pixels from the CCD. A computer can reconstruct the image by varying the light intensity for each spot on the computer monitor in the proper order. As is well known in the art, such digital images can be stored on disk, transmitted over a computer network and analyzed using powerful image processing techniques. Any two-dimensional detector or CCD can be used. A variety of CCDs and two-dimensional detectors are available commercially (e.g., from Hamamatsu Corp. of Bridgewater, N.J.). A variety of automated imaging systems that combine CCDs with computers and image processing software are also available commercially (e.g., the ARRAYWORXS™ microarray scanner available from Applied Precision, Inc. of Issaquah, Wash.).

In one embodiment, the fluorescent or chemiluminescent light is detected by scanning the microarray of the present invention. An apparatus using the scanning method of detection collects light data from the sample relative to a detection device by moving either the microarray or the detection device. Preferably, the microarray is scanned by moving the detection device. When two different fluorescent or chemiluminescent indicators need to be resolved, the light from the microarray may be passed thought a single monochromator, a grating or a prism. Alternatively, filters could be used to resolve the colors spectrally. For the scanning method of detection, the detector is preferably a diode array which records the light that is emitted at a particular spatial position. As is well known in the art, software can then be used to recreate the scanned image, resulting in a single image containing the entire microarray of the invention. As described above, such digital images can be stored on disk, transmitted over a computer network and analyzed using very powerful image processing techniques.

### EXAMPLE 1

### Preparation of Microarrays of Polymeric Biomaterials

A 25 mm by 75 mm epoxy modified glass microscope slide (available from Xenopore Corp. of Hawthorne, N.J. as XENOSLIDE™ E) was coated with polyHEMA (available from Sigma) by dipping it in a 75 mg/ml polyHEMA solution in 95% ethanol for a few seconds and allowing the surface to dry overnight at room temperature.

Stock solutions of the sixteen synthetic polymers listed in Table 1 (available from Sigma or Boehringer Ingelheim Corp. of Ridgefield, Conn.), each containing 50 mg/ml polymer in dimethylformamide (available from Sigma), were prepared.

From these sixteen stock solutions, mixtures of the 120 pairwise synthetic polymer combinations in ratios of 90:10, 50:50 and 10:90 were also prepared. Taken together, the 16 original stock solutions and 360 mixtures formed a first set of 376 stock solutions.

A first slide was prepared by depositing small drops of this first set of 376 stock solutions in the form of an 8×47 rectangular microarray on a coated microscope slide using a SYNQUAD 5500™ liquid handling robot equipped with eight ChipMaker2™ pins arranged in a single row. These pins have a split quill design, take up by capillary action a sample volume of about 250 nl from the separate reservoirs of the robotic liquid handling device, and deposit a spot volume of about 1 nl, having a diameter of between 100 and 200 μm. After depositing one drop for each of the elements of the microarray, the process was repeated four more times in such a way that a total of five identical drops were deposited to form each element of the microarray. In order to minimize cross contamination between the different polymeric compositions, the row of pins was washed in dimethylformamide in between each of the 235 (i.e., 5×47) deposition steps.

TABLE 1

| Polymer | Comments |
|---|---|
| Poly(ethylene adipate) | Average MW 10,000 |
| Poly(ethylene adipate) | Average MW 1,000, OH terminated |
| Poly(1,3-propylene succinate) | Average MW 9,500 |
| Poly(1,3-propylene glutarate) | Average MW 7,100 |
| Poly(1,3-propylene adipate) | Average MW 5,700 |
| Poly(1,4-butylene adipate) | Average MW 12,000 |
| Poly(1,4-butylene adipate) | Average MW 1,000, OH terminated |
| Poly(D,L-lactic acid) | MW 20,000 to 30,000 |
| Poly(D,L-lactide-co-caprolactone) | lac:cap ratio 40:60 |
| Poly(D,L-lactide-co-caprolactone) | lac:cap ratio 85:15 |
| Poly(D,L-lactide-co-glycolide) | lac:gly ratio 50:50, |
| | MW 40,000 to 75,000 |
| Poly(D,L-lactide-co-glycolide) | lac:gly ratio 65:35, |
| | MW 40,000 to 75,000 |
| Poly(D,L-lactide-co-glycolide) | lac:gly ratio 75:25, |
| | MW 90,000 to 126,000 |
| Poly(D,L-lactide-co-glycolide) | lac:gly ratio 85:15, |
| | MW 90,000 to 126,000 |
| Poly(D,L-lactide-co-glycolide) | lac:gly ratio 50:50, |
| | average MW 40,000 |
| Poly(D,L-lactide-co-glycolide) | lac:gly ratio 50:50, |
| | average MW 30,000 |

Second and third sets of 376 stock solutions comprising poly(D,L-lactide) and 40:60 poly(D,L-lactide-co-caprolactone), respectively, mixed separately in a 50:50 ratio with each member of the first set of 376 stock solutions were also prepared, and deposited on second and third slides, respectively, in the form of 8×47 rectangular microarrays as described above for the first slide.

The slides were then dried under vacuum in a desiccator at 25° C. for 2 days before use to remove any residual dimethylformamide. FIG. 2 is a photograph of an 8×47 microarray in which individual polymer spots having a diameter of between 100 and 200 μm were deposited at 375 μm intervals.

### EXAMPLE 2

### Immunofluorescence of Collagen II in Chondrocyte Cells

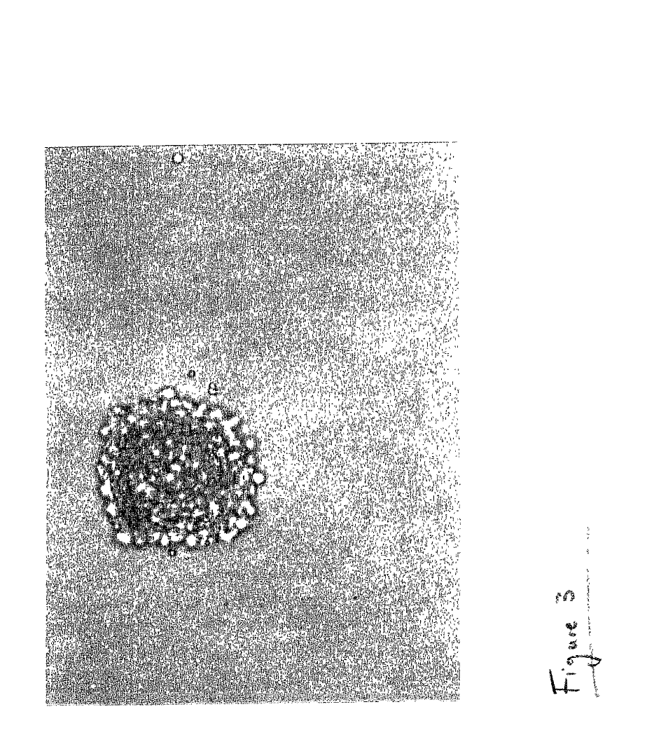

A microarray of polymeric biomaterials prepared according to Example 1 was washed for 5 minutes with complete bovine growth medium. It was then placed in a 25 mm by 150 mm round suspension culture dish and seeded with a solution of bovine chondrocyte cells that had been incubated in complete bovine growth medium at 37° C. for 5 days. The growth medium was changed daily, and the cells were allowed to grow for 7 days at 37° C. FIG. 3 is a phase contrast image of bovine chondrocyte cells growing on a single spot of a seeded microarray.

The growth medium was then removed and the seeded microarray slide cleared of non-adhered cells by washing with phosphate buffered saline (PBS). The adhered cells were then fixed by soaking the slide in 10% (v/v) formalin for 4 minutes. The slide was washed for about 10 minutes with heat-inactivated 1.5% normal goat serum (available from Vector Laboratories, Inc. of Burlingame, Calif.) in PBS and for 10 minutes with PBS alone.

In order to facilitate entry of the antibodies into the fixed cells and in order to minimize non-specific binding of the secondary goat antibodies, the slide was incubated at 25° C. for 30 minutes in a solution containing 0.2% (v/v) of the non-ionic surfactant Triton X-100™ (available from Sigma) and 10% (v/v) goat serum in PBS.

The slide was then incubated at 25° C. for 2 hours in a primary antibody solution containing 10 μg/ml rabbit anti-collagen II antibody (available from Rockland Inc. of Gilbertsville, Pa.) in PBS and 1.5% (v/v) goat serum. As a control, a second seeded microarray slide was incubated in a PBS and 1.5% (v/v) goat serum solution that lacked the primary antibody. In order to remove any unbound primary antibody, the slides were washed for 10 minutes with 1.5% (v/v) goat serum in PBS; for 10 minutes with 1.5% (v/v) goat serum and 0.2% (v/v) TRITON X-100™ in PBS; and for a further 10 minutes with 1.5% (v/v) goat serum in PBS.

The slides were then incubated at 25° C. for 1.5 hours in a secondary antibody solution containing 10 μg/ml goat anti-rabbit antibody labeled with an oregon green marker (available from Rockland Inc. of Gilbertsville, Pa.) in PBS and 1.5% (v/v) goat serum. In order to remove any unbound secondary antibody, the slides were washed for 10 minutes with 1.5% (v/v) goat serum in PBS and for a further 30 minutes with PBS alone.

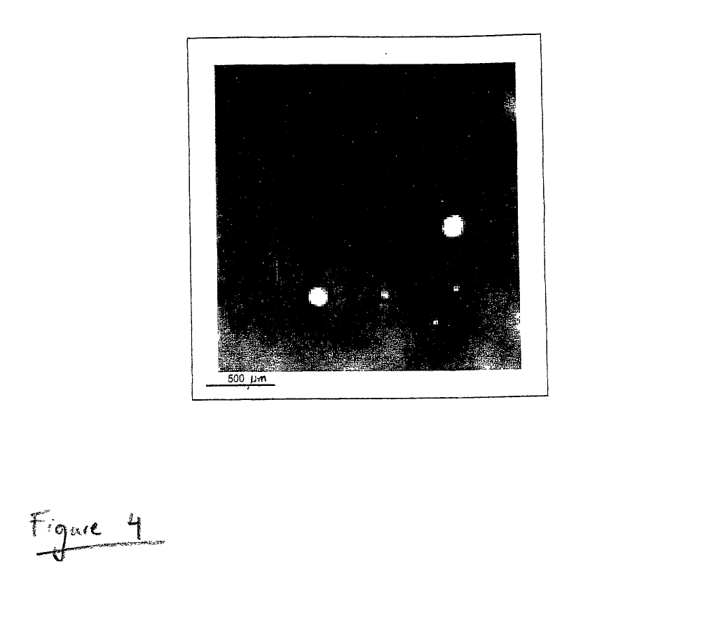

Finally, after applying a few drops of mounting medium (available as VECTAMOUNT™ from Vector Laboratories, Inc. of Burlingame, Calif.), placing a 22 mm by 60 mm coverslip on the slide and sealing the edges, the microarray was imaged using an ARRAYWORXS™ microarray scanner (available from Applied Precision, Inc. of Issaquah, Wash.). FIG. 4 is a photograph of such a scan in which bovine chondrocyte cells were grown on 150 μm polymer spots. White spots represent significant levels of oregon green and hence of collagen II. The control slide that lacked primary rabbit antibody showed no sign of oregon green, suggesting the absence of non-specific binding by the secondary goat antibody.

### EXAMPLE 3

### Immunofluorescence of Neurofilament in Neural Stem Cells

A microarray of polymeric biomaterials prepared according to Example 1 was washed with complete DMEM growth medium. It was then placed in a 25 mm by 150 mm round suspension culture dish and seeded with a solution of neural stem cells that had been incubated in complete growth medium at 37° C. for 4 days. The growth medium was changed daily, and the cells were allowed to grow for 7 days at 37° C.

The immunostaining procedure was as described for bovine chondrocyte cells in Example 2, except that rabbit anti-neurofilament primary antibodies (available from Chemicon International of Temicula, Calif.) were used with goat anti-rabbit secondary antibodies labeled with fluorescein (available from Jackson ImmunoResearch Laboratories Inc., of West Grove, Pa.). FIG. 5 is a photograph of such immunostained neural stem cells grown on 150 μm polymer spots.

### EXAMPLE 4

### Expression of a Gene of Interest in Chondrocytes

A microarray of polymeric biomaterials prepared according to Example 1 was seeded with chondrocytes as in Example 2, except that during initial incubation, the cells were additionally transfected with a plasmid containing the promoter sequence of a gene of interest fused to a luciferase reporter gene (available from Promega Corp. of Madison, Wis.). See Current Protocols in Molecular Biology, Ed. by Ausubel et al., John Wiley & Sons Inc., New York, N.Y., 2000 for transfection protocols.

The growth medium was removed and the seeded microarray slide was cleared of non-adhered-cells by washing with PBS. The microarray was then flooded with luciferase substrate (available as beetle luciferin from Promega, Corp. of Madison, Wis.) in a biological buffer solution (pH 7.8) containing 20 mM tricine, 0.1 mM EDTA, 33 mM dithiothreitol (DTT), 0.3 mM coenzyme A, 0.5 mM ATP, and 1 mM MgCl₂. In the presence of the luciferase substrate, cells that contained the luciferase protein generated light by chemiluminescence. The light signals were then used to identify those polymeric biomaterials that caused attached chondrocytes to express the gene of interest.

Other embodiments of the invention will be apparent to those skilled in the art from a consideration of the specification or practice of the invention disclosed herein. It is intended that the specification and examples be considered as exemplary only, with the true scope of the invention being indicated by the following claims.

We claim:

1. A microarray of polymeric biomaterials comprising:
a base comprising a cytophobic surface; and
a plurality of discrete polymeric biomaterial elements non-covalently bound to said cytophobic surface.

2. A microarray of polymeric biomaterials comprising:
a base comprising a cytophobic surface; and
a plurality of discrete non-monolayer polymeric biomaterial elements bound to said cytophobic surface.

3. The microarray of claim 1 or 2, wherein said base comprises a material selected from the group consisting of glass, plastic, metal, ceramic, and combinations thereof.

4. The microarray of claim 1 or 2, wherein said cytophobic surface comprises a hydrogel.

5. The microarray of claim 4, wherein said hydrogel comprises a polymer selected from the group consisting of homopolymers of methacrylic acid esters, homopolymers of alkylene oxides, homopolymers of alkylene glycols, copolymers thereof, and mixtures thereof.

6. The microarray of claim 4, wherein said hydrogel comprises a polymer selected from the group consisting of poly(methyl methacrylate), poly(isobutyl methacrylate), poly(pentyl methacrylate), poly(2-hydroxy-ethyl methacrylate), copolymers thereof, and mixtures thereof.

7. The microarray of claim 4, wherein said hydrogel comprises a polymer selected from the group consisting of poly(ethylene oxide), poly(propylene 1,2-glycol), poly(propylene 1,3-glycol), copolymers thereof, and mixtures thereof.

8. The microarray of claim 1, wherein said polymeric biomaterial elements are bound to said cytophobic surface via a non-covalent interaction selected from the group consisting of chemical adsorption, hydrogen bonding, surface interpenetration, ionic bonding, van der Waals forces, hydrophobic interactions, magnetic interactions, dipole-dipole interactions, and combinations thereof.

9. The microarray of claim 2, wherein said polymeric biomaterial elements are bound to said cytophobic surface via an interaction selected from the group consisting of chemical adsorption, hydrogen bonding, surface interpenetration, covalent bonding, ionic bonding, van der Waals forces, hydrophobic interactions, magnetic interactions, dipole-dipole interactions, and combinations thereof.

10. The microarray of claim 1 or 2, wherein each of said polymeric biomaterial elements comprises at least one polymer selected from the group consisting of synthetic polymers, adducts thereof, and mixtures thereof.

11. The microarray of claim 10, wherein said synthetic polymers are selected from the group consisting of polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, poly(vinyl chloride), poly(vinyl fluoride), poly(vinyl imidazole), poly(vinyl alcohol), and chlorosulphonated polyolefins.

12. The microarray of claim 10, wherein at least one of said polymeric biomaterial elements further comprises a compound selected from the group consisting of drugs, growth factors, combinatorial compounds, proteins, polysaccharides, polynucleotides, lipids, adducts thereof, and mixtures thereof.

13. The microarray of claim 12, wherein said compound is covalently bound to the synthetic polymer component or components of the polymeric biomaterial.

14. The microarray of claim 12, wherein said compound is non-covalently bound to the synthetic polymer component or components of the polymeric biomaterial.

15. The microarray of claim 1 or 2, wherein each of said polymeric biomaterial elements are between 10 and 1000 μm in diameter.

16. The microarray of claim 1 or 2, wherein each of said polymeric biomaterial elements are between 50 and 500 μm in diameter.

17. The microarray of claim 1 or 2, wherein said polymeric biomaterial elements are disposed at between 100 and 1200 μm intervals in a rectangular microarray.

18. The microarray of claim 1 or 2, wherein said polymeric biomaterial elements are disposed at between 300 and 500 μm intervals in a rectangular microarray.

19. The microarray of claim 1 or 2, wherein said polymeric biomaterial elements are present at a density on said cytophobic surface that ranges from 1 to 1,000 polymeric biomaterial elements per cm².

20. The microarray of claim 1 or 2, wherein said polymeric biomaterial elements are present at a density on said cytophobic surface that ranges from 10 to 100 polymeric biomaterial elements per cm².

21. A method for the high throughput screening of polymeric biomaterials for their ability to affect cellular behavior comprising:
providing a microarray of polymeric biomaterial elements that are bound to a cytophobic surface;
contacting said microarray with a cell culture for a period of time sufficient to allow the cells to adhere to said polymeric biomaterial elements; and
assaying the cellular behavior for each polymeric biomaterial element of the microarray.

22. The method of claim 21, wherein said cytophobic surface comprises a hydrogel.

23. The method of claim 22, wherein said hydrogel comprises a polymer selected from the group consisting of homopolymers of methacrylic acid esters, homopolymers of alkylene oxides, homopolymers of alkylene glycols, copolymers thereof, and mixtures thereof.

24. The method of claim 22, wherein said hydrogel comprises a polymer selected from the group consisting of poly(methyl methacrylate), poly(isobutyl methacrylate), poly(pentyl methacrylate), poly(2-hydroxy-ethyl methacrylate), copolymers thereof, and mixtures thereof.

25. The method of claim 22, wherein said hydrogel comprises a polymer selected from the group consisting of poly(ethylene oxide), poly(propylene 1,2-glycol), poly(propylene 1,3-glycol), copolymers thereof, and mixtures thereof.

26. The method of claim 21, wherein said polymeric biomaterial elements are non-covalently bound to said cytophobic surface.

27. The method of claim 26, wherein said polymeric biomaterial elements are bound to said cytophobic surface via a non-covalent interaction selected from the group consisting of chemical adsorption, hydrogen bonding, surface interpenetration, ionic bonding, van der Waals forces, hydrophobic interactions, magnetic interactions, dipole-dipole interactions, and combinations thereof.

28. The method of claim 21, wherein said polymeric biomaterial elements are not monolayers.

29. The method of claim 21, wherein each of said polymeric biomaterial elements comprises at least one polymer selected from the group consisting of synthetic polymers, adducts thereof, and mixtures thereof.

30. The method of claim 29, wherein said synthetic polymers are selected from the group consisting of polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, poly(vinyl chloride), poly(vinyl fluoride), poly(vinyl imidazole), poly(vinyl alcohol), and chlorosulphonated polyolefins.

31. The method of claim 29, wherein at least one of said polymeric biomaterial elements further comprises a compound selected from the group consisting of drugs, growth factors, combinatorial compounds, proteins, polysaccharides, polynucleotides, lipids, adducts thereof, and mixtures thereof.

32. The method of claim 31, wherein said compound is covalently bound to the synthetic polymer component or components of the polymeric biomaterial.

33. The method of claim 31, wherein said compound is non-covalently bound to the synthetic polymer component or components of the polymeric biomaterial.

34. The method of claim 21, wherein said polymeric biomaterial elements are between 10 and 1000 μm in diameter.

35. The method of claim 21, wherein said polymeric biomaterial elements are between 50 and 500 μm in diameter.

36. The method of claim 21, wherein:
said microarray is a rectangular microarray; and
said polymeric biomaterial elements are disposed at between 100 and 1200 μm intervals on said cytophobic surface.

37. The method of claim 21, wherein:
said microarray is a rectangular microarray; and
said polymeric biomaterial elements are disposed at between 300 and 500 μm intervals on said cytophobic surface.

38. The method of claim 21, wherein said polymeric biomaterial elements are present at a density on said cytophobic surface that ranges from 1 to 1,000 polymeric biomaterial elements per cm².

39. The method of claim 21, wherein said polymeric biomaterial elements are present at a density on said cytophobic surface that ranges from 10 to 1000 polymeric biomaterial elements per cm².

40. The method of claim 21, wherein said cells are selected from the group consisting of mammalian cells, bacterial cells, yeast cells, and plant cells.

41. The method of claim 21, wherein said cells are selected from the group of mammalian cells consisting of chondrocytes, fibroblasts, connective tissue cells, epithelial cells, endothelial cells, cancer cells, hepatocytes, islet cells, smooth muscle cells, skeletal muscle cells, heart muscle cells, kidney cells, intestinal cells, organ cells, lymphocytes, blood vessel cells, stem cells, human embryonic stem cells, and mesenchymal stem cells.

42. The method of claim 21, wherein the step of assaying comprises assaying for cellular proliferation.

43. The method of claim 21, wherein the step of assaying comprises assaying for cellular differentiation.

44. The method of claim 21, wherein the step of assaying comprises assaying for gene expression.

45. A method of preparing a microarray of polymeric biomaterials comprising:
providing a base comprising a substrate surface;
providing polymeric biomaterials in a solvent selected from the group consisting of dimethylformamide, dimethylsulfoxide, chloroform, and dichlorobenzene; and
depositing said polymeric biomaterials as a plurality of discrete elements on said substrate surface using a robotic liquid handling device, wherein
said polymeric biomaterials are dissolved at a concentration of between 10 and 200 mg/ml in said solvent, and said substrate surface comprises a hydrogel.

46. The method of claim 45, wherein said liquid handling device deposits via pin fluid deposition.

47. The method of claim 45, wherein said liquid handling device deposits via syringe pumped fluid deposition.

48. The method of claim 45, wherein said liquid handling device deposits via piezoelectric fluid deposition.

49. The method of claim 45, wherein said polymeric biomaterial elements are deposited as drops of between 0.1 and 100 nl.

50. The method of claim 45, wherein said polymeric biomaterial elements are deposited as drops of between 1 and 10 nl.

51. A method for the high throughput screening of compounds for their ability to affect cellular behavior comprising:
providing a microarray of polymeric biomaterial elements arranged on a cytophobic surface;
contacting said polymeric biomaterial elements with a cell culture for a period of time sufficient to allow the cells to adhere to said polymeric biomaterial elements; and
assaying the cellular behavior for each polymeric biomaterial element of the microarray, wherein:
at least one of said polymeric biomaterial elements comprises one of said compounds.

52. The method of claim 51, wherein said compounds are drugs.

53. The method of claim 51, wherein said compounds belong to a synthetic combinatorial library of compounds

54. The method of claim 51, wherein said compounds are selected from the group consisting of proteins, polysaccharides, polynucleotides, lipids, adducts thereof, and mixtures thereof.
